(19) United States
(12) Patent Application Publication
(10) Pub. No.: US 2005/0008643 A1
(43) Pub. Date: Jan. 13, 2005

(54) Diagnostic methods and agents

(76) Inventors: Ora Bernard, North Balwyn (AU); Victoria Caitlin Foletta, Malvern (AU)

Correspondence Address: Leopold Presser;Scully Scott Murphy & Presser, 400 Garden City Plaza, Garden City, NY, 11530, US

(21) Appl. No.: 10/481,849

(22) PCT Filed: Jun. 27, 2002
(86) PCT No.: PCT/AU02/00834

Related U.S. Application Data
(60) Provisional application No. 60/330,361, filed on Oct. 18, 2001.

(30) Foreign Application Priority Data
Jun. 27, 2001 (AU) PR5965

(51) Int. Cl. A61K 39/395

(52) U.S. Cl. 424/146.1; 424/94.5

(57) ABSTRACT

The present invention relates generally to a method for detecting an aberrant cell in a subject or in a biological sample from said subject and agents useful for same. The presence of the aberrant cell or group of aberrant cells provides an indication of a particular disease or condition or a propensity for development of a disease or condition. More particularly, the present invention contemplates a method for detecting a cell associated with cancer or having a propensity to develop into a cancer cell in a subject or in a biological sample from said subject by determining the relative increase in the presence of a LIM kinase protein or a related enzyme or a relative increase in LIM kinase activity or a relative increase in the presence of expression products from a gene encoding a LIM kinase or a related protein. The present invention further provides a method for diagnosing the presence of a cancer or cancerous-like growth or distinguishing between an invasive and non-invasive cancer in a subject or in a biological sample from said subject by screening for up-regulation of a LIM kinase or a related protein in a cell or group of cells or an up-regulation in the presence of expression products of genetic sequences encoding a LIM kinase or a related protein. The present invention provides diagnostic agents useful for detecting LIM kinase or expression products of genetic material encoding LIM kinase. Such diagnostic agents include immunointeractive molecules, such as antibodies, and genetic probes for detecting expression products of LIM kinase genes. The present invention further provides genetically modified animals exhibiting altered levels of LIM kinase. Such animals are useful models for screening for anti-cancer agents.

### FIELD OF THE INVENTION

The present invention relates generally to a method for detecting an aberrant cell in a subject or in a biological sample from said subject and agents useful for same. The presence of the aberrant cell or group of aberrant cells provides an indication of a particular disease or condition or a propensity for development of a disease or condition. More particularly, the present invention contemplates a method for detecting a cell associated with cancer or having a propensity to develop into a cancer cell in a subject or in a biological sample from said subject by determining the relative increase in the presence of a LIM kinase protein or a related enzyme or a relative increase in LIM kinase activity or a relative increase in the presence of expression products from a gene encoding a LIM kinase or a related protein. The present invention further provides a method for diagnosing the presence of a cancer or cancerous-like growth or distinguishing between an invasive and non-invasive cancer in a subject or in a biological sample from said subject by screening for up-regulation of a LIM kinase or a related protein in a cell or group of cells or an up-regulation in the presence of expression products of genetic sequences encoding a LIM kinase or a related protein. The present invention provides diagnostic agents useful for detecting LIM kinase or expression products of genetic material encoding LIM kinase. Such diagnostic agents include immunointeractive molecules, such as antibodies, and genetic probes for detecting expression products of LIM kinase genes. The present invention further provides genetically modified animals exhibiting altered levels of LIM kinase. Such animals are useful models for screening for anti-cancer agents.

### BACKGROUND OF THE INVENTION

Bibliographic details of the publications referred to in this specification are collected at the end of the description.

Reference to any prior art in this specification is not, and should not be taken as, an acknowledgment or any form of suggestion that this prior art forms part of the common general knowledge in any country.

Cancer is one of the most debilitating disease conditions affecting humans and the incidence and prevalence of cancer is increasing. A number of risk factors are associated with the development of cancer or with the likelihood of development of cancer and these include genetic predispositions, familial forms of cancer and environmental factors. The environment is constantly changing and populations are exposed to high levels of potentially toxic compounds on a daily basis. The increasing affluence of populations and societies also means expansion of industrial endeavours and many of these industries use potentially toxic compounds.

There is a need, therefore, to provide to rapid detection of cancer cells and to use this information to effect early diagnosis of and clinical intervention in the treatment or prophylaxis of the cancer.

A number of cancer markers have been proposed. However, as the threat of cancer increases and as the population grows older, it is important to search for new markers which may be more efficacious or accurate in a cancer assay. Furthermore, it is significant that there is currently no diagnostic test for ovarian cancer. As a result, this cancer is usually metastatic when identified and this is then often too late for interventionist or preventionist therapy.

In work leading up to the present invention, the inventors investigated LIM kinase. This enzyme contains two LIM motifs at the N-terminal portion of the molecule. Two forms of LIM kinase are known, LIM kinase 1 and LIM kinase 2. LIM kinase is important in regulating actin dynamics and hence has a role in cell division, development and migration. The present inventors have now determined that LIM kinase is up-regulated in cancer cells and in particular invasive cancer cells relative to normal cells or non-invasive cancer cells. This provides a useful marker for the diagnosis of cancer by immunological and genetic assays and permits development of cancer targeting agents for cancer imaging and to generate anti-cancer agents.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the determination that LIM kinase is up-regulated in cancer cells and in particular invasive cancer cells relative to normal or non-invasive cancer cells. LIM kinase is an enzyme involved in the regulation of actin dynamics and is particularly important for cell division, development and migration. The identification of a cancer-specific marker permits development of a range of diagnostic agents, including cancer imaging agents and cancer targeting agents having therapeutic applications.

Accordingly, the present invention in one aspect contemplates a method for detecting an aberrant cell in a subject or in a biological sample from the subject by contacting cells, cell extracts, serum or other sample from the subjects or said biological sample with an immunointeractive molecule specific for a LIM kinase or antigenic portion thereof and screening for the level of immunointeractive molecule-LIM kinase complex formation wherein an elevated presence of the complex relative to a normal cell is indicative of an aberrant cell.

In an alternative embodiment, the aberrant cell is detected at the genetic level by screening for the level of an expression of a gene encoding a LIM kinase wherein an elevated level of the expression product compared to a normal cell is indicative of an aberrant cell. Real-time PCR as well as other PCR procedures are useful for determining transcriptional activity. Elevated levels of LIM kinase gene transcriptional activity is proposed to be indicative of cancer or a propensity for development of cancer.

These methods are useful inter alia for diagnosing the presence of cancer or cancer-like growth in a subject. For example, cells or cell extracts are screened immunologically for the presence of elevated levels of LIM kinase. Alternatively, mRNA is obtained from cells of a subject or from a biological sample from a subject and cDNA optionally generated. The mRNA or cDNA is then contacted with a genetic probe capable of hybridizing to and/or amplifying all or part of a nucleotide sequence encoding LIM kinase or its complementary nucleotide sequence and then the level of the mRNA or cDNA is detected wherein the presence of elevated levels of the mRNA or cDNA compared to normal controls is indicative of the presence of cancer.

These tests are not only useful for diagnosing the presence of a primary cancer but also for assessing the risk of remission based on monitoring residual cancer cells.

The present invention further contemplates diagnostic and therapeutic agents. In one embodiment, the present invention provides a deimmunized antibody molecule having specificity for an epitope recognized by a monoclonal antibody to LIM kinase wherein at least one of the CDRs of the variable domain of the deimmunized antibody is derived from the the monoclonal antibody to LIM kinase and the remaining immunoglobulin-derived parts of the deimmunized antibody molecule are derived from an immunoglobulin or an analog thereof from the host for which the antibody is to be deimmunized.

The deimmunized antibody may be used, for example, for cancer imaging. In one embodiment, the method comprises introducing into a patient a deimmunized form of a non-human derived monoclonal antibody specific for human LIM kinase or an antigenic determinant thereon labeled with a reporter molecule, allowing dissemination of the labeled antibody throughout the circulatory system, or to selected parts of the circulatory system and then subjecting the patient to reporter molecule-detection means to identify the location of the antibody.

Yet another aspect of the present invention contemplates a method for the treatment of a patient having cancer by administering to the human a cancer cell growth inhibiting-effective amount of an antibody having specificity for human LIM kinase wherein the antibody is substantially non-immunogenic and further comprises a cell growth inhibiting or cell killing agent fused, bound or otherwise associated thereto. The method may alternatively involve the use of genetic means to inhibit LIM kinase gene expression. The present invention may also extend to the use of modulators of LIM kinase activity to inhibit LIM kinase and reduce cancer cell growth. Some conditions may also benefit from promoting cell growth by up-regulating LIM kinase activity. Reference to “modulators” of LIM kinase activity include agents which act at the protein level or the level of LIM kinase gene expression or post-transcriptional processes. The present invention contemplates, therefore, compositions comprising the cancer targeting agents of the present invention and one or more pharmaceutically acceptable carriers and/or diluents. The compositions may also comprise modulators of LIM kinase gene expression or protein activity.

Yet another aspect of the present invention contemplates the use of a monoclonal antibody to LIM kinase in the manufacture of a quantitative or semi-quantitative diagnostic kit to determine relative levels of LIM kinase in suspected cancer cells from a patient. The kit may come with instructions for use and may be automated or semi-automated or in a form which is compatible with automated machine or software.

### BRIEF DESCRIPTION OF THE FIGURES

FIG. 1 is a photographic representation of a Western blot analysis of LIM kinase (LIMK) protein expression in various normal, non-metastatic and metastatic tissue culture cell lines. Top panel: Detection of LIMK using rat anti-LIMK monoclonal antibody. Bottom panel: Detection of common heat shock protein (HSP-70) to demonstrate protein loading using mouse monoclonal anti-HSP-70 antibody. Lanes: 1. NIH 3T3 fibroblasts; 2. Ras-transformed NIH 3T3 fibroblast 3. MCF-7 (non-metastatic breast cancer cell line); 4. LNCap (non-metastatic prostate cancer cell line); 5.PC-3 (metastatic prostate cancer cell line6. MDA MB 231 (metastatic breast cancer cell line).

FIG. 2 is a photographic representation of Western blot analysis of LIMK1 expression showing LIMK1 I protein in human metastatic melanoma and ovarian cancer tissues in comparison with cell lines. Endogenous LIMK1 expression in 50 μg of protein lysates extracted from metastatic ovarian cancer tissues (lanes 1-7), metastatic melanoma (lanes 9-12) and cancer cell lines (lanes 13-15).

FIG. 3 is a photographic representation showing immunohistochemical staining for LIMK1 protein in human metastatic tumors and normal tissues. Paraffin embedded tissues were stained with anti-LIMK1 antibody (A-E) or isotype-match control (F). (A) Normal lung tissue, (B), metastatic lung cancer (C) Normal breast tissue, (D) metastatic breast cancer tissue, (E) metastatic prostate cancer and (E) isotype-matched control.

### DETAILED DESCRIPTION OF THE PRESENT INVENTION

The present invention is predicated in part on the determination that aberrant cells and in particular cancer cells and even more particularly invasive cancer cells or cells with a propensity for developing into cancer cells elevated levels of LIM kinase relative to normal cells or non-invasive cancer cells. The latter enzyme is a protein kinsae and is involved inter alia in early embryonic development patterning and in regulating actin dynamics. A cell which produces high levels of LIM kinase or high levels of LIM kinase activity is deemed, in accordance with the present invention, to be an aberrant cell and a cell associated with or likely to be associated with a disease or disorder such as cancer or a related condition. The ability to detect a LIM kinase or expression products of genetic material encoding a LIM kinase provides a means for detecting or diagnosing cancer or a propensity for the development of cancer or a related condition and a means of distinguishing between invasive and non-invasive cancers. Furthermore, modulators such as antagonists of LIM kinase activity may be useful in inhibiting or reducing cancer cell growth.

Accordingly, one aspect of the present invention contemplates a method for detecting an aberrant cell in a subject or in a biological sample from said subject, said method comprising contacting cells, cell extracts or serum or other sample from said subject or said biological sample with an immunointeractive molecule specific for a LIM kinase or antigenic portion thereof and screening for the level of immunointeractive molecule-LIM kinase complex formations wherein an elevated presence of said complex relative to a normal cell is indicative of an aberrant cell.

The “sample” above includes a biological sample from the subject such as circulatory fluid.

In a related embodiment, the present invention provides a method for detecting an aberrant cell in a subject or in a biological sample from said subject, said method comprising screening the level of an expression product of a gene encoding a LIM kinase wherein an elevated level of said expression product compared to a normal cell is indicative of an aberrant cell.

Reference herein to a “subject” includes an animal including a mammal such as a human, primate, laboratory test animal (e.g. mouse, rabbit, rat, guinea pig), livestock animal (e.g. sheep, cow, pig, horse) or companion animal (e.g. cat, dog). The preferred subject is a human.

A “biological sample” from a subject includes a biopsy and may be any sample of cells, cell extract, tissue, tissue fluid, excretia, circulatory fluid or respiratory fluid or other material. Reference to “circulatory fluid” includes blood, serum and lymph fluid. The biological sample may be extracted, treated, untreated, diluted or concentrated from the subject.

Reference to a “normal” cell includes a cell not regarded as aberrant or cancerous and may be considered an “average” of normal cell types. A comparison of LIM kinase levels may also be made to a non-invasive cancer cell.

The “immunointeractive molecule” is any molecule having specificity and binding affinity for LIM kinase or its antigenic parts or its homologs or derivatives. Although the preferred immunointeractive molecule is an immunglobulin molecule, the present invention extends to other immunointeractive molecules such as antibody fragments, single chain antibodies, deimmunized including humanized antibodies and T-cell associated antigen-binding molecules (TABMs). Most preferably, the immunointeractive molecule is an antibody such as a polyclonal or monoclonal antibody. Most preferably, the antibody is a monoclonal antibody.

The immunointeractive molecule exhibits specificity for LIM kinase or more particularly an antigenic determinant or epitope on LIM kinase. An antigenic determinant or epitope on LIM kinase includes that part of the molecule to which an immune response is directed. The antigenic determinant or epitope may be a B-cell epitope or where appropriate a T-cell epitope. The term “antigenic part” includes an antigenic determinant or epitope.

An “expression product” is generally mRNA or cDNA and the amount of expression product provides an indicator of the level of LIM kinase gene expression and provides, therefore, indirect evidence for the presence of LIM kinase. Conveniently, pools of mRNA or cDNA are obtained or cell extracts comprising total mRNA obtained and genetic probes complementary to all or part of the LIM kinase gene-specific mRNA or cDNA. Binding of probes may then be quantitated or semi-quantitated.

Reference herein to LIM kinase includes reference to both LIM kinase 1 and LIM kinase 2 or their homologs or derivatives.

Reference to a “level” of LIM kinase includes an amount quantitatively, semi-quantitatively or qualitatively determined.

In accordance with the present invention, it is proposed that cells associated with a cancer including cancer cells and in particular invasive cancer cells produce elevated levels of LIM kinase. The quantitative or qualitative detection of levels of LIM kinase or expression products of genetic material encoding LIM kinase provides, therefore, an indicator that the cell is aberrant and is associated with cancer or has a propensity to develop into a cancer.

Detection of elevated levels of LIM kinase even after initial treatment is important to assess the likelihood of remission. Early detection and/or monitoring of residual cancer cells assists in developing therapeutic protocols to treat or to target LIM kinase producing cells after initial treatment to thereby reduce the risk of remission.

Accordingly, another aspect of the present invention contemplates a method for diagnosing the presence of cancer or cancer-like growth in a subject, said method comprising contacting cells or cell extracts from said subject or a biological sample from said subject with a LIM kinase-binding effective amount of an antibody having specificity for said LIM kinase or an antigenic determinant or epitope thereon and then quantitatively or qualitatively determining the level of a LIM kinase-antibody complex wherein the presence of elevated levels of said complex compared to a normal cell is indicative of the presence of a cancer.

In a related embodiment, the present invention provides a method for diagnosing the presence of a cancer in a subject, said method comprising obtaining mRNA from cells of said subject or from a biological sample from said subject and optionally generating cDNA and contacting said mRNA or cDNA with a genetic probe capable of hybridizing to and/or amplifying all or part of a nucleotide sequence encoding LIM kinase or its complementary nucleotide sequence and then detecting the level of said mRNA or cDNA wherein the presence, of elevated levels of said mRNA or cDNA compared to normal controls is indicative of the presence of cancer.

These aspects of the present invention may also be applied to distinguishing between invasive and non-invasive cancers. In that case, the levels of LIM kinase is compared to a non-invasive cancer cell.

The use of antibodies and in particular monoclonal antibodies to detect LIM kinase is the preferred method of the present invention. Antibodies may be prepared by any of a number of means. For the detection of human LIM kinase, antibodies are generally but not necessarily derived from non-human animals such as primates, livestock animals (e.g. sheep, cows, pigs, goats, horses), laboratory test animals (e.g. mice, rats, guinea pigs, rabbits) and companion animals (e.g. dogs, cats). Generally, antibody based assays are conducted in vitro on cell or tissue biopsies. However, if an antibody is suitably deimmunized or, in the case of human use, humanized, then the antibody can be labeled with, for example, a nuclear tag, administered to a patient and the site of nuclear label accumulation determined by radiological techniques. The LIM kinase antibody is regarded, therefore, as a cancer targeting agent. Accordingly, the present invention extends to deimmunized forms of the antibodies for use in cancer imaging in human and non-human patients. This is described further below.

The present invention provides, therefore, an antibody and in particular a monoclonal antibody for use in immunological assays for LIM kinase or for cancer imaging in vivo.

For the generation of antibodies to a LIM kinase, the enzyme is required to be extracted from a biological sample whether this be from animal including human tissue or from cell culture if produced by recombinant means. The LIM kinase can be separated from the biological sample by any suitable means. For example, the separation may take advantage of any one or more of LIM kinase's surface charge properties, size, density, biological activity and its affinity for another entity (e.g. another protein or chemical compound to which it binds or otherwise associates). Thus, for example, separation of LIM kinase from the biological fluid may be achieved by any one or more of ultra-centrifugation, ion-exchange chromatography (e.g. anion exchange chromatography, cation exchange chromatography), electrophoresis (e.g. polyacrylamide gel electrophoresis, isoelectric focussing), size separation (e.g., gel filtration, ultra-filtration) and affinity-mediated separation (e.g. immunoaffinity separation including, but not limited to, magnetic bead separation such as Dynabead (trademark) separation, immunochromatography, immuno-precipitation). Choice of the separation technique(s) employed may depend on the biological activity or physical properties of the particular LIM kinase sought or from which tissues it is obtained.

Preferably, the separation of LIM kinase from the biological fluid preserves conformational epitopes present on the kinase and, thus, suitably avoids techniques that cause denaturation of the enzyme. Persons of skill in the art will recognize the importance of maintaining or mimicking as close as possible physiological conditions peculiar to the LIM kinase (e.g. the biological fluid from which it is obtained) to ensure that the antigenic determinants or active site/s on the LIM kinase, which are exposed to the animal, are structurally identical to that of the native enzyme. This ensures the raising of appropriate antibodies in the immunized animal that would recognize the native enzyme. In a preferred embodiment, the kinase is separated from the biological fluid using any one or more of affinity separation, gel filtration and ultra-filtration.

Immunization and subsequent production of monoclonal antibodies can be carried out using standard protocols as for example described by Köhler and Milstein (Kohler and Milstein, Nature 256: 495-499, 1975; Kohler and Milstein, Eur. J. Immunol. 6(7): 511-519, 1976), Coligan et al. (“Current Protocols in Immunology, John Wiley & Sons, Inc., 1991-1997) or Toyama et al. (Monoclonal Antibody, Experiment Manual”, published by Kodansha Scientific, 1987). Essentially, an animal is immunized with a LIM kinase-containing biological fluid or fraction thereof or a recombinant form of LIM kinase by standard methods to produce antibody-producing cells, particularly antibody-producing somatic cells (e.g. B lymphocytes). These cells can then be removed from the immunized animal for immortalization.

Where a fragment of LIM kinase is used to generate antibodies, it may need to first be associated with a carrier. By “carrier” is meant any substance of typically high molecular weight to which a non- or poorly immunogenic substance (e.g. a hapten) is naturally or artificially linked to enhance its immunogenicity.

Immortalization of antibody-producing cells may be carried out using methods which are well-known in the art. For example, the immortalization may be achieved by the transformation method using Epstein-Barr virus (EBV) (Kozbor et al., Methods in Enzymology 121: 140, 1986). In a preferred embodiment, antibody-producing cells are immortalized using the cell fusion method (described in Coligan et al., 1991-1997, supra), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. Mice spleen cells are particularly useful. It would be possible, however, to use rat, rabbit, sheep or goat cells, or cells from other animal species instead.

Specialized myeloma cell lines have been developed from lymphocytic tumours for use in hybridoma-producing fusion procedures (Kohler and Milstein, 1976, supra; Shulman et al., Nature 276: 269-270, 1978; Volk et al., J. Virol. 42(1): 220-227, 1982). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumour cells to produce their own antibodies. To eliminate the production of tumour cell antibodies by the hybridomas, myeloma cell lines incapable of producing endogenous light or heavy immunoglobulin chains are used. A third reason for selection of these cell lines is for their suitability and efficiency for fusion.

Many myeloma cell lines may be used for the production of fused cell hybrids, including, e.g. P3×63-Ag8, P3×63-AG8.653, P3/NS1-Ag4-1 (NS-1), Sp2/0-Ag14 and S194/5.XXO.Bu.1. The P3×63-Ag8 and NS-1 cell lines have been described by Köhler and Milstein (1976, supra). Shulman et al. (1978, supra) developed the Sp2/0-Ag14 myeloma line. The S194/5.XXO.Bu.1 line was reported by Trowbridge (J. Exp. Med. 148(1): 313-323, 1978).

Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Kohler and Milstein, 1975, supra; Kohler and Milstein, 1976, supra; Gefter et al., Somatic Cell Genet. 3: 231-236, 1977; Volk et al., 1982, supra). The fusion-promoting agents used by those investigators were Sendai virus and polyethylene glycol (PEG).

Because fusion procedures produce viable hybrids at very low frequency (e.g. when spleens are used as a source of somatic cells, only one hybrid is obtained for roughly every 1×10⁵ spleen cells), it is preferable to have a means of selecting the fused cell hybrids from the remaining unfused cells, particularly the unfused myeloma cells. A means of detecting the desired antibody-producing hybridomas among other resulting fused cell hybrids is also necessary. Generally, the selection of fused cell hybrids is accomplished by culturing the cells in media that support the growth of hybridomas but prevent the growth of the unfused myeloma cells, which normally would go on dividing indefinitely. The somatic cells used in the fusion do not maintain long-term viability in in vitro culture and hence do not pose a problem. In the example of the present invention, myeloma cells lacking hypoxanthine phosphoribosyl transferase (HPRT-negative) were used. Selection against these cells is made in hypoxanthine/aminopterin/thymidine (HAT) medium, a medium in which the fused cell hybrids survive due to the HPRT-positive genotype of the spleen cells. The use of myeloma cells with different genetic deficiencies (drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Kennet et al. (Monoclonal Antibodies and Hybridomas: A New Dimension in Biological Analyses, pp 376-384, Plenum Press, New York, 1980) and by FACS analysis (O'Reilly et al., Biotechniques 25: 824-830, 1998).

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumours that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines are tested for their specificity to detect the LIM kinase of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target LIM kinase but which does not recognize non-target epitopes are identified and then directly cultured in vitro or injected into a histocompatible animal to form tumours and to produce, collect and purify the required antibodies.

These antibodies are LIM kinase specific. This means that the antibodies are capable of distinguishing LIM kinase from other molecules. More broad spectrum antibodies may be used provided that they do not cross react with molecules in a normal cell. One particularly useful LIM kinase-specific antibody is described. The above procedure is applicable to LIM kinase 1 or 2.

Where the monoclonal antibody is destined for use in in vivo cancer imaging, it will need to be deimmunized with respect to the host into which it will be introduced (e.g. a human). The deimmunization process may take any of a number of forms including the preparation of chimeric antibodies which have the same or similar specificity as the monoclonal antibodies prepared according to the present invention. Chimeric antibodies are antibodies whose light and heavy chain genes have been constructed, typically by genetic engineering, from immunoglobulin variable and constant region genes belonging to different species. Thus, in accordance with the present invention, once a hybridoma producing the desired monoclonal antibody is obtained, techniques are used to produce interspecific monoclonal antibodies wherein the binding region of one species is combined with a non-binding region of the antibody of another species (Liu et al., Proc. Natl. Acad. Sci. USA 84: 3439-3443, 1987). For example, complementary determining regions (CDRs) from a non-human (e.g. murine) monoclonal antibody can be grafted onto a human antibody, thereby “humanizing” the murine antibody (European Patent No. 0 239 400; Jones et al., Nature 321: 522-525, 1986; Verhoeyen et al., Science 239: 1534-1536, 1988; Richmann et al., Nature 332: 323-327, 1988). In this case, the deimmunizing process is specific for humans. More particularly, the CDRs can be grafted onto a human antibody variable region with or without human constant regions. The non-human antibody providing the CDRs is typically referred to as the “donor” and the human antibody providing the framework is typically referred to as the “acceptor”. Constant regions need not be present, but if they are, they must be substantially identical to human immunoglobulin constant regions, i.e. at least about 85-90%, preferably about 95% or more identical. Hence, all parts of a humanized antibody, except possibly the CDRs, are substantially identical to corresponding parts of natural human immunoglobulin sequences. Thus, a “humanized antibody” is an antibody comprising a humanized light chain and a humanized heavy chain immunoglobulin. A donor antibody is said to be “humanized”, by the process of “humanization”, because the resultant humanized antibody is expected to bind to the same antigen as the donor antibody that provides the CDRs. Reference herein to “humanized” includes reference to an antibody deimmunized to a particular host, in this case, a human host.

It will be understood that the deimmunized antibodies may have additional conservative amino acid substitutions which have substantially no effect on antigen binding or other immunoglobulin functions. Exemplary conservative substitutions may be made according to Table 1.

TABLE 1

| ORIGINAL | EXEMPLARY |
|---|---|
| RESIDUE | SUBSTITUTIONS |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Exemplary methods which may be employed to produce deimmunized antibodies according to the present invention are described, for example, in Richmann et al., 1988, supra; European Patent No. 0 239 400; U.S. Pat. No. 6,056,957, U.S. Pat. No. 6,180,370, U.S. Pat. No. 6,180,377.

Thus, in one embodiment, the present invention contemplates a deimmunized antibody molecule having specificity for an epitope recognized by a monoclonal antibody to LIM kinase wherein at least one of the CDRs of the variable domain of said deimmunized antibody is derived from the said monoclonal antibody to LIM kinase and the remaining immunoglobulin-derived parts of the deimmunized antibody molecule are derived from an immunoglobulin or an analog thereof from the host for which the antibody is to be deimmunized.

This aspect of the present invention involves manipulation of the framework region of a non-human antibody.

The present invention extends to mutants and derivatives of the subject antibodies but which still retain specificity for LIM kinase.

The terms “mutant” or “derivatives” includes one or more amino acid substitutions, additions and/or deletions.

As used herein, the term “CDR” includes CDR structural loops which covers to the three light chain and the three heavy chain regions in the variable portion of an antibody framework region which bridge β strands on the binding portion of the molecule. These loops have characteristic canonical structures (Chothia et al., J. Mol. Biol. 196: 901, 1987; Chothia et al., J. Mol. Biol. 227: 799, 1992).

By “framework region” is meant region of an immunoglobulin light or heavy chain variable region, which is interrupted by three hypervariable regions, also called CDRs. The extent of the framework region and CDRs have been precisely defined (see, for example, Kabat et al., “Sequences of Proteins of Immunological Interest”, U.S. Department of Health and Human Sciences, 1983). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of LIM kinase.

As used herein, the term “heavy chain variable region” means a polypeptide which is from about 110 to 125 amino acid residues in length, the amino acid sequence of which corresponds to that of a heavy chain of a monoclonal antibody of the invention, starting from the amino-terminal (N-terminal) amino acid residue of the heavy chain. Likewise, the term “light chain variable region” means a polypeptide which is from about 95 to 130 amino acid residues in length, the amino acid sequence of which corresponds to that of a light chain of a monoclonal antibody of the invention, starting from the N-terminal amino acid residue of the light chain. Full-length immunoglobulin “light chains” (about 25 Kd or 214 amino acids) are encoded by a variable region gene at the NH₂-terminus (about 110 amino acids) and a κ or λ constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g. γ (encoding about 330 amino acids).

The term “immunoglobulin” or “antibody” is used herein to refer to a protein consisting of one or more polypeptides substantially encoded by immunoglobulin genes. The recognized immunoglobulin genes include the κ. λ, α. γ (IgG₁, IgG₂, IgG₃, IgG₄), δ. ε and μ constant region genes, as well as the myriad immunoglobulin variable region genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, Fab′ and (Fab′)₂.

The invention also contemplates the use and generation of fragments of monoclonal antibodies produced by the method of the present invention including, for example, Fv, Fab, Fab′ and F(ab′)₂ fragments. Such fragments may be prepared by standard methods as for example described by Coligan et al. (1991-1997, supra).

The present invention also contemplates synthetic or recombinant antigen-binding molecules with the same or similar specificity as the monoclonal antibodies of the invention. Antigen-binding molecules of this type may comprise a synthetic stabilized Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement.

Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Krebber et al. (J. Immunol. Methods 201(1): 35-55,1997). Alternatively, they may be prepared by methods described in U.S. Pat. No. 5,091,513, European Patent No 239,400 or the articles by Winter and Milstein (Nature 349: 293, 1991) and Plückthun et al. (In Antibody engineering: A practical approach, 203-252, 1996).

Alternatively, the synthetic stabilized Fv fragment comprises a disulphide stabilized Fv (dsFv) in which cysteine residues are introduced into the V_{H} and V_{L} domains such that in the fully folded Fv molecule the two residues will form a disulphide bond therebetween. Suitable methods of producing dsFv are described, for example, in (Glockshuber et al., Biochem. 29: 1363-1367, 1990; Reiter et al., J. Biol. Chem. 269: 18327-18331, 1994; Reiter et al., Biochem. 33: 5451-5459, 1994; Reiter et al., Cancer Res. 54: 2714-2718, 1994; Webber et al., Mol. Immunol. 32: 249-258, 1995).

Also contemplated as synthetic or recombinant antigen-binding molecules are single variable region domains (termed dAbs) as, for example, disclosed in (Ward et al., Nature 341: 544-546, 1989; Hamers-Casterman et al., Nature 363: 446-448, 1993; Davies & Riechmann, FEBS Lett. 339: 285-290, 1994).

Alternatively, the synthetic or recombinant antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the synthetic or recombinant antigen binding molecule may comprise non-immunoglobulin derived, protein frameworks. For example, reference may be made to (Ku & Schutz, Proc. Natl. Acad. Sci. USA 92: 6552-6556, 1995) which discloses a four-helix bundle protein cytochrome b562 having two loops randomized to create CDRs, which have been selected for antigen binding.

The synthetic or recombinant antigen-binding molecule may be multivalent (i.e. having more than one antigen binding site). Such multivalent molecules may be specific for one or more antigens. Multivalent molecules of this type may be prepared by dimerization of two antibody fragments through a cysteinyl-containing peptide as, for example disclosed by (Adams et al., Cancer Res. 53: 4026-4034, 1993; Cumber et al., J. Immunol. 149: 120-126, 1992). Alternatively, dimerization may be facilitated by fusion of the antibody fragments to amphiphilic helices that naturally dimerize (Plünckthun, Biochem 31: 1579-1584, 1992) or by use of domains (such as leucine zippers jun and fos) that preferentially heterodimerize (Kostelny et al., J. Immunol. 148: 1547-1553, 1992). Multivalent antibodies are useful, for example, in detecting different forms of LIM kinase of a LIM kinase and another cancer marker.

The present invention further encompasses chemical analogs of amino acids in the subject antibodies. The use of chemical analogs of amino acids is useful inter alia to stabilize the molecules such as if required to be administered to a subject. The analogs of the amino acids contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogs. Analogs of LIM kinase or fragments or derivatives thereof are also contemplated as potential antagonists or agonists (i.e. modulators) of LIM kinase activity.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylburyrate | Mgabu | L-N-methylarginine | Nmarg |
| aminocyclopropanecarboxy- | Cpro | L-N-methylasparagine | Nmasn |
| late | | L-N-methylaspartic acid | Nmasp |
| aminoisobutyric acid | Aib | L-N-methylcysteine | Nmcys |
| aminonorbornylcarboxy- | Norb | L-N-methylglutamine | Nmgln |
| late | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

The present invention further contemplates an assay to detect LIM kinase including the steps of:
(1) contacting a monoclonal antibody specific to LIM kinase or an antigenic determinant thereon with a biological sample suspected of containing a cell containing said LIM kinase; and
(2) subjecting the complex formed in step (1) to a signal detection step.

The signal detection step may include ELISA or any other reporter molecule based assays. As part of this detection step, the signal may first need to be amplified.

A deimmunized monoclonal antibody of the present invention may also be useful for cancer imaging in vivo as well as for targeting cancer cells in order to bring the cancer cells into contact with cell growth retarding or cell killing agents, i.e. cytostatic or cytocidal agents.

With respect to cancer imaging, a reporter molecule is attached to the deimmunized monoclonal antibody and this is then introduced to a host, such as a human. By detecting the reporter molecule, cancer growths can be visualized. One particularly useful form of reporter molecule is a nuclear tag.

Accordingly, another aspect of the present invention contemplates a method for detecting cancer cells in a human patient, said method comprising introducing into said patient a deimmunized form of a non-human derived monoclonal antibody specific for human LIM kinase or an antigenic determinant thereon labeled with a reporter molecule, allowing dissemination of the labeled antibody throughout the circulatory system, or to selected parts of the circulatory system and then subjecting said patient to reporter molecule-detection means to identify the location of the antibody.

Immunological based LIM kinase detection protocols may take a variety of forms. For example, a plurality of antibodies may be immobilized in an array each with different specificities to particular antigens or cancer cells including LIM kinase. Cells from a biopsy are then brought into contact with the antibody array and a diagnosis may be made as to the type of cancer based on the cells which are immobilized.

Other more conventional assays may also be conducted such as by ELISA, Western blot analysis, immunoprecipitation analysis, immunofluorescence analysis, immunochemistry analysis or FACS analysis.

The present invention provides, therefore, a method of detecting in a sample a LIM kinase or fragment, variant or derivative thereof comprising contacting the sample with an antibody or fragment or derivative thereof and detecting the level of a complex comprising said antibody and LIM kinase or fragment, variant or derivative thereof compared to normal controls wherein elevated levels of LIM kinase is indicative of cancer growth.

As discussed above, any suitable technique for determining formation of the complex may be used. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to Coligan et al., 1991-1997, supra which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labeled antigen-binding molecule to a target antigen. The antigen in this case is LIM kinase or a fragment thereof.

Two-site assays are particularly favoured for use in the present invention. A number of variations of these assays exist, all of which are intended to be encompassed by the present invention. Briefly, in a typical forward assay, an unlabeled antigen-binding molecule such as an unlabeled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labeled with a reporter molecule capable of producing a detectable signal is then added and incubated, allowing time sufficient for the formation of another complex of antibody-antigen-labeled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labeled antibody are added simultaneously to the bound antibody. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions' to allow binding of any antigen-present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labeled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labeled with a reporter molecule. Depending on the amount of target and the strength of the reporter molecule signal, a bound antigen may be detectable by direct labelling with the antibody. Alternatively, a second labeled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:
(a) direct attachment of the reporter molecule to the antibody;
(b) indirect attachment of the reporter molecule to the antibody; i.e., attachment of the reporter molecule to another assay reagent which subsequently binds to the antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by International Patent Publication No. WO 93/06121. Reference also may be made to the fluorochromes described in U.S. Pat. Nos. 5,573,909 and 5,326,692. Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent-labeled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.

Monoclonal antibodies to LIM kinase may also be used in ELISA-mediated detection of LIM kinase especially in serum or other circulatory fluid. This may be undertaken in any number of ways such as immobilizing anti-LIM kinase antibodies to a solid support and contacting these with a biological extract such as serum, blood, lymph or other bodily fluid, cell extract or cell biopsy. Labeled anti-LIM kinase antibodies are then used to detect immobilized LIM kinase. This assay may be varied in any number of ways and all variations are encompassed by the present invention. This approach enables rapid detection and quantitation of LIM kinase levels using, for example, a serum-based assay.

In another embodiment, the method for detection comprises detecting the level of expression in a cell of a polynucleotide encoding a LIM kinase. Expression of said polynucleotide may be determined using any suitable technique. For example, a labeled polynucleotide encoding a LIM kinase may be utilized as a probe in a Northern blot of an RNA extract obtained from the cell. Preferably, a nucleic acid extract from the animal is utilized in concert with oligonucleotide primers corresponding to sense and antisense sequences of a polynucleotide encoding the kinase, or flanking sequences thereof, in a nucleic acid amplification reaction such as RT PCR. A variety of automated solid-phase detection techniques are also appropriate. For example, a very large scale immobilized primer arrays (VLSIPS (trademark)) are used for the detection of nucleic acids as, for example, described by Fodor et al. (Science 251: 767-777, 1991) and Kazal et al. (Nature Medicine 2: 753-759, 1996). The above genetic techniques are well known to persons skilled in the art.

For example, to detect LIM kinase encoding RNA transcripts, RNA is isolated from a cellular sample suspected of containing LIM kinase RNA, e.g. total RNA isolated from human cancer tissue. RNA can be isolated by methods known in the art, e.g. using TRIZOL (trademark) reagent (GIBCO-BRL/Life Technologies, Gaithersburg, Md.). Oligo-dT, or random-sequence oligonucleotides, as well as seuqence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al. (Quant. Biol. 51: 263, 1987; Erlich, eds., PCR Technology, Stockton Press, NY, 1989). Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian LIM kinase genes. For example, one primer is prepared which is predicted to anneal to the antisense strand and another primer prepared which is predicted to anneal to the sense strand of a cDNA molecule which encodes a LIM kinase.

To detect the amplified product, the reaction mixture is typically subjected to agarose gel electrophoresis or other convenient separation technique and the relative presence of the LIM kinase specific amplified DNA detected. For example, LIM kinase amplified DNA may be detected using Southern hybridization with a specific oligonucleotide probe or comparing is electrophoretic mobility with DNA standards of known molecular weight. Isolation, purification and characterization of the amplified LIM kinase DNA may be accomplished by excising or eluting the fragment from the gel (for example, see references Lawn et al., Nucleic Acids Res. 2: 6103, 1981; Goeddel et al., Nucleic cids Res. 8: 4057-1980), cloning the amplified product into a cloning site of a suitable vector, such as the pCRII vector (Invitrogen), sequencing the cloned insert and comparing the DNA sequence to the known sequence of LIM kinase. The relative amounts of LIM kinase mRNA and cDNA can then be determined.

Real-time PCR is particularly useful in determining transcriptional levels of PCR genes. Determination of transcriptional activity also includes a measure of potential translational activity based on available mRNA transcripts. Real-time PCR as well as other PCR procedures use a number of chemistries for detection of PCR product including the binding of DNA binding fluorophores, the 5′ endonuclease, adjacent liner and hairpin oligoprobes and the self-fluorescing amplicons. These chemistries and real-time PCR in general are discussed, for example, in Mackay et al., Nucleic Acids Res 30(6): 1292-1305, 2002; Walker, J. Biochem. Mol. Toxicology 15(3): 121-127, 2001; Lewis et al., J. Pathol. 195: 66-71, 2001.

The present invention may be used to detect any cancer which comprises cells which express elevated levels of LIM kinase. Generally, the cancer would be invasive rather than non-invasive.

The subject method may be used to detect hyperplastic/neoplastic cells of hematopoietic origin, e.g. arising from myeloid, lymphoid or erythroid lineages or precursor cells thereof. For example, the present invention encompasses the detection of various myeloid disorders including but not limited to acute promyeloid leukemia (APML), acute myelogenous leukemia (AML) and chronic myelogenous leukemia (CML). Lymphoid malignancies which may be detected by the subject method include but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Wodenstrom's macroglobulinemia (WM). Additional forms of malignant lymphomas detectable by the method of the present invention include but are not limited to non-Hodgkin's lymphoma and variants thereof, peripheral T-cell lymphomas, adult T-cell leukemia/lymphoma (ATL), cutaneous T-cell lymphona (CTCL), large granular lymphcytic leukemia (LGF) and Hodgkin's disease.

The subject method can also be used to detect malignancies of the various organ systems, such as those affecting lung, breast, lymphoid, gastrointestinal and genito-urinary tract as well as adenocarcinomas which include malignancies such as most colon cancers, renal-cell carcinoma, prostate cancer and/or testicular tumours, non-small cell carcinoma of the lung, cancer of the small intestine and lancer of the esophagus. Solid tumours that can be detected according to the method of the present invention include sarcomas and carcinomas such as but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medullobalstoma, craniopharyngioma, ependymoma, melanoma, pinealoma, hemangioblastoma, acoustic neuroma, oliogdendroglioma, meningioma, melanoma, neuroblastoma and retinoblastoma. Ovarian cancer, breast cancer and melanoma detection is particularly preferred.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms “neoplasia” and “hyperplasia” can be used interchangeably, referring generally to cells experiencing abnormal cell growth rates. Neoplasis and hyperplasis include “tumors” which may be either benign, pre-malignant or malignant.

As used herein, the terms “hyperproliferative” and “neoplastic” are used interchangeably and refer to those cells in an abnormal state or condition characterized by rapid proliferation or neoplasm. The terms are meant to include all types of cancerous growths or oncogenic processes, metastatic tissues or malignantly transformed cells, tissues or organs irrespective of histopathologic type or state of invasiveness. “Pathologic hyperproliferative” cells occur in disease states characterized by malignant tumor growth.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatis carcinomas, endocrine system carcinomas and melanomas. Exexmplary carcinomas include those forming from tissue of the cervix, lung, prostate, breast, head and neck, colon and ovary. The term also includes carcinosarcomas, e.g. which include malignant tumors composed of carcinomatous and sarcomatous tissues. An “adenocarcinoma” refers to a carcinoma derived from glandular tissue or in which the tumor cells form recognizable glandular structures.

The identification of LIM kinase as a cancer-specific molecule permits the generation of targeting agents to destroy or at least retard the growth of the cancer cells. In particular, the cancer targeting agents comprising LIM kinase specific antibodies are fused, bound or otherwise associated with a cell growth inhibiting or killing agent. Such agents include but are not limited to cytocidal or cytostatic agents which act at the protein or corresponding mRNA or DNA levels. For example, the cell growth or killing agent maybe a nuclear tag or may be an agent which promotes induction of antagonists of LIM kinase RNAi or RNA oligonucleotides.

Accordingly, another aspect of the present invention contemplates a method for the treatment of a patient having cancer, said method comprising administering to said human, a cancer cell growth inhibiting-effective amount of an antibody having specificity for human LIM kinase and being substantially non-immunogenic and further comprising a cell growth inhibiting or cell killing agent fused, bound or otherwise associated thereto.

In an alternative embodiment, the present invention provides a method of treating a patient having cancer or a related condition, said method comprising the administration to said patient of a LIM kinase-inhibiting effective amount of an agent for a time and under conditions sufficient to inhibit the activity of LIM kinase or reduce levels of LIM kinase and to reduce cancer cell growth.

The agent is an example of a modulator of LIM kinase activity. Such an agent may act on the LIM kinase itself or on the expression of the LIM kinase gene or LIM kinase mRNA. The agent may be a derivative of LIM kinase such as a chemical analog or be identified following natural product screening or the screening of chemical libraries. In some circumstances, the modulator may be an agonist in situations where cell growth is to be promoted.

The present invention further contemplates compositions comprising agents capable of acting as modulators of LIM kinase activity or gene expression and one or more pharmaceutically acceptable carriers and/or diluents.

The pharmaceutical forms suitable for injectable use include sterile aqueous solutions. It must be stable under the conditions of manufacture and storage and must be preserved against the contaminating action of microorganisms such as bacteria and fingi. The carrier can be a solvent or dilution medium comprising, for example, water, ethanol, polyol (for example, glycerol, propylene glycol and liquid polyethylene-glycol, and the like), suitable mixtures thereof and vegetable oils. The proper fluidity can be maintained, for example, by the use of superfactants. The preventions of the action of microorganisms can be brought about by various anti-bacterial and anti-fungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with the active ingredient and optionally other active ingredients as required, followed by filtered sterilization or other appropriate means of sterilization.

Pharmaceutically acceptable carriers and/or diluents include any and all solvents, dispersion media, coatings, anti-bacterial and anti-fungal agents, isotonic and absorption delaying agents and the like. The use of such media and agents for pharmaceutical active substances is well known in the art and except insofar as any conventional media or agent is incompatible with the active ingredient, their use in the therapeutic compositions is contemplated. Supplementary active ingredients can also be incorporated into the compositions. In an alternative embodiment, the present invention contemplates genetic constructs such as comprising antisense, sense and ribozyme constructs or RNAi or RNA oligonucleotides. Such genetic compositions are also referred to as DNA vaccination compositions and are specifically directed to modulating expression at the transcriptional or translation levels of nucleotide sequences encoding a LIM kinase.

Accordingly, another aspect of the present invention contemplates a method for the treatment of a patient having cancer, said method comprising administering to said human, a genetic composition comprising a genetic construct which down-regulates expression of a gene encoding LIM kinase.

Reference herein to “expression” includes down-regulating the steps transcription, translation or both. Particularly preferred genetic constructs are antisense constructs to LIM kinase, induce co-suppression of the LIM kinase gene or induce RNAi-mediated down-regulation of LIM kinase mRNA transcript.

The present invention further contemplates the use of a monoclonal antibody to LIM kinase in the manufacture of a quantitative or semi-quantitative diagnostic kit to determine relative levels of LIM kinase in suspected cancer cells from a patient. The kit may come with instructions for use and may be automated or semi-automated or in a form which is compatible with automated machine or software.

The generation of antibodies to LIM kinase may, in accordance with the present invention, be directed to the active or inactive forms of the molecule. Antibodies directed to an active LIM kinase, i.e. activated by phosphorylation of threonine or its equivalent in the activation loop of the kinase domain, are particularly useful in detecting an increase or decrease in LIM kinase activity. The present invention extends, therefore, to antibodies to differentially phosphorylated LIM kinases and their use in screening for agents which inhibit LIM kinase activity.

The present invention is further described by the following non-limiting Examples.

### EXAMPLE 1

### Detection of Elevated LIM Kinase in Cancer Cells

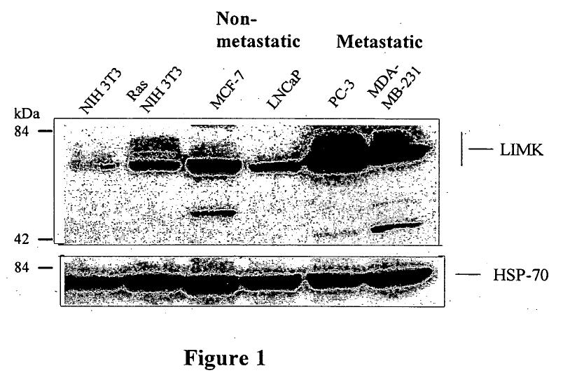

Rat monoclonal antibodies to LIM kinase were generated and used in a Western blot analysis of LIM kinase (LIMK) in various normal and cancer cell lines. To control the level of loading in each well, the cells were also probed with murine monoclonal anti-HSP-70 antibody. The results are shown in FIG. 1.

LIMK protein expression was detected in all tissue culture cells examined. Low levels were detected in the non-transformed cell lines 293T (lane 3) and NIH-3T3 (lane 4). Moderate levels were expressed by mouse olfactory epithelial cells (4.4.2; lane 1). Ras-transformed NIH 3T3 fibroblasts (lane 5); non-metastatic human breast epithelial carcinoma (MCF-7; lane 6); non-metastatic human prostate carcinoma (LNCap; lane 7) and transformed monkey kidney epithelial cells (COS-7; lane 2). High levels of LIMK are expressed by metastatic human prostate carcinoma (PC-3; lane 8) and metastatic human breast carcinoma (MDA MB 231).

The results show that LIM kinase is up-regulated in cancer cells in comparison with normal cells and is expressed at even higher levels in metastatic cancers and is, therefore, a marker for the development of non-metastatic and metastatic cancers. It should also be noted that cancer cells contain high levels of activated LIMK (the higher molecular weight bands) in comparison with non-cancer cells.

### EXAMPLE 2

### Over-Expression of LIMK Induces Invasiveness in Non-Metastatic Breast Cancer Cells

Expression constructs comprising the genetic sequence encoding LIMK1 are cloned into the pCDNA vector and transfected into the non-metastatic human breast epithelial carcinoma cell line, MCF-7, using standard techniques. Vectors comprising expression constructs having one, two or three copies of the LIMK1-encoding sequence are generated. For comparison, cells are also transfected with vectors comprising vRhoA and Δ4ROCK. MCF-7 cells transfected with vector alone are not especially invasive, infiltrating fewer than 1,000 cells. This accords with the known non-metastatic nature of these cells. When transfected with a single, two or three copies of LIMK1, however, MCF-7 cells are increasingly more invasive.

### EXAMPLE 3

### LIMK Increases Invasiveness of Metastatic Human Breast Cancer Cells

The effect of LIMK1 on the extent of invasion of metastatic human cancer cells is assayed. Vectors comprising or not comprising LIMK1-encoding sequences or the dominant-negative variant thereof are transfected into the metastatic human breast cancer cell line, MDA-MB-231, using standard techniques. Vectors comprising expression constructs having one, two or three copies of the LIMK1-encoding sequence or the dominant-negative (DN) variant thereof are generated and used. Extent of infiltration by LIMK1-transfected, DN-LIMK1-transfected and vector-transfected breast cancer cells are then assayed on Matrigel chambers.

Metastatic MDA-MB-231 cells transfected with vector alone are more invasive than the non-metastatic cell line MCF-7. When transfected with a single, two or three copies of LIMK1, MDA-MB-231 cells are increasingly more invasive.

Transfection of MDA-MB-231 cells with the LIMK DN variant reverse their invasiveness.

### EXAMPLE 4

### Metastases of MDA-MB-231 Breast Cancer Cells into Bone In Vivo

The extent of invasion of LIMK1-expressing human cancer cells in vivo is demonstrated. Nude mice are given intra-cardiac injections of MDA-MB-231 human breast cancer cells, which are transfected with vector alone or vector comprising the genetic sequence encoding LIMK1 or vector comprising the genetic sequence encoding a dominant negative (DN) variant of LIMK1.

After three weeks, animals are sacrificed and analyzed. Results indicate that metastases are twice as high in animals that receive cells transfected with the wild-type LIMK1-encoding sequence, compared to those that received vector alone. Moreover, in animals that receive cells transfected with the vector comprising the dominant-negative variant, metastasis is virtually eliminated. This extent of metastases is matched to a concomitant analysis of the number of lesions and their sizes in each case.

In addition, the skeletal structure of mice is examined and the extent of invasion into the bone structure of the mice is determined by visualization of LIMK1 through detection of a rat monoclonal antibody specific for LIMK. This confirms that LIMK1 in MDA-MB-231 cells expressing LIMK1 metastasize throughout the skeletal structure of the mice.

### EXAMPLE 5

### Expression of LIM Kinase in Human Cancer Tissue Biopsies

The confirmation of LIM kinase as a predictive test for metastatic and non-metastatic cancers in humans is confirmed using biopsy material from a range of cancer cell types, including inter alia breast, prostate, kidney and colon.

Tissue samples (at least 100, for each indication) are collected from hospitalized cancer patients and frozen for subsequent analysis. Each tissue sample is examined for expression of LIM kinase, using one or both of two methods: western blot analysis and ELISA.

(a) Western Blot Analysis

Total proteins, from frozen biopsy tissue samples, are extracted and quantified. A protein aliquot (50 μg) is run on 10% w/v polyacrylamide gel electrophoresis and transferred onto nitrocellulose filter, using standard techniques. Filters are then probed with rat anti-LIM kinase monoclonal antibody to visualize LIM kinase.

(b) ELISA

Total proteins, from frozen biopsy tissue samples or serum or blood, are extracted and quantified. LIMK1 expression is then visualized using an ELISA test, following standard procedures.

### EXAMPLE 6

### Effects of Expression of LIM Kinase in Human Prostate Cancer Cells In Vitro

Expression constructs comprising the genetic sequence encoding LIMK1 are cloned into pEGFP vector and are transfected into the non-metastatic human prostate carcinoma cell lines, LNCap and C-4, using standard techniques. Vectors comprising expression constructs having one, two or three copies of the LIMK1-encoding sequence are generated. As a control, transfections of empty vector are also carried out.

Simultaneously, vector alone and vector comprising LIMK1-encoding sequence are also transfected into metastatic human prostate carcinoma cell lines, PC-3 and DU-145. In this case, vectors comprising the sequence encoding the dominant negative LIMK variant are also employed.

In all cases, three cell lines for each treatment and one for the respective controls are selected for further analysis on Matrigel chambers.

The results will show that non-metastatic LNCap and C-4 cells, transfected with vector alone, are not especially invasive. When transfected with a single, two or three copies of LIMK1, however, LNCap and C-4 cells are increasingly more invasive, as did breast cancer cells.

By comparison, the metastatic cell lines PC-3 and DU-145, when transfected with a single, two or three copies of LIMK1, display even greater invasiveness than those transfected solely with empty vector. As with breast cells, transfection of metastatic PC-3 and DU-145 cells with the LIMK DN variant reverses their invasiveness.

### EXAMPLE 7

### Metastases of Human Prostate Cancer Cells In Vivo

The extent of invasion of LIMK1-expressing human prostate cancer cells in vivo is also determined, in an analagous way as was carried out for breast cancer cells. Nude mice are given intra-cardiac injections of selected human prostate cancer cells, which have been transfected with vector alone, or vector comprising the genetic sequence encoding LIMK1, or vector comprising the genetic sequence encoding a dominant-negative variant of LIMK1.

After three weeks, animals are sacrificed and analyzed. Results will indicate that metastatic extent is higher in animals that receive cells transfected with the wild-type LIMK1-encoding sequence, compared with those that receive vector alone. Moreover, in animals that received cells transfected with the vector comprising the dominant-negative variant, metastasis is virtually eliminated. Examination of the skeletal structure of mice, and the extent of invasion into the bone, is determined via visualization of LIMK1 through detection of a rat monoclonal antibody specific for LIMK1.

### EXAMPLE 8

### Real-Time PCR

LIM kinase gene expression may be used to determine transcriptional activity. Elevated transcriptional activity may be expected in cancer cells. Real-time PCR is a particularly convenient means of determining gene expression. Methods for real-time PCR may be found in Mackay et al., 2002, supra, Walker, 2001, supra and Lewis et al., 2001, supra. Detection of elevated transcriptional activity is indicative of cancer or a propensity for the development of cancer.

### EXAMPLE 9

### Detection of elevated LIMK1 in Metatastic Melanomas and Ovarian Cancer Tissues

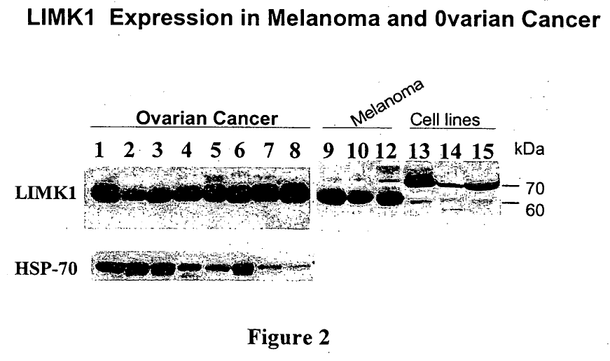

Extremely high levels of LIMK1 expression were detected in human melanoma and ovarian cancer tissues in comparison with cell lines and normal tissues using Western blot analysis. The levels of LIMK1 in these tissues is estimated to be as high as 0.1% of the total proteins in these tissues. The results are shown in FIG. 2.

### EXAMPLE 10

### Immunochemistry Analysis of LIMK1 Expression in Metatastic Prostate, Lung and Breast Cancer Tissue

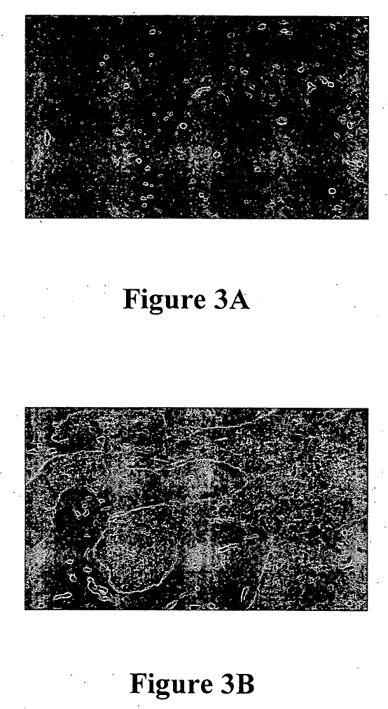

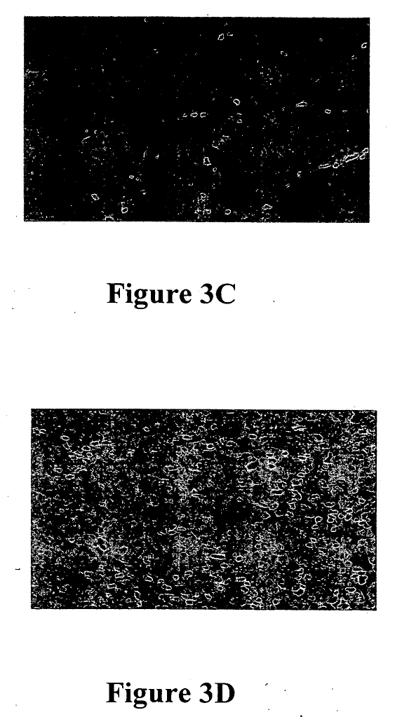

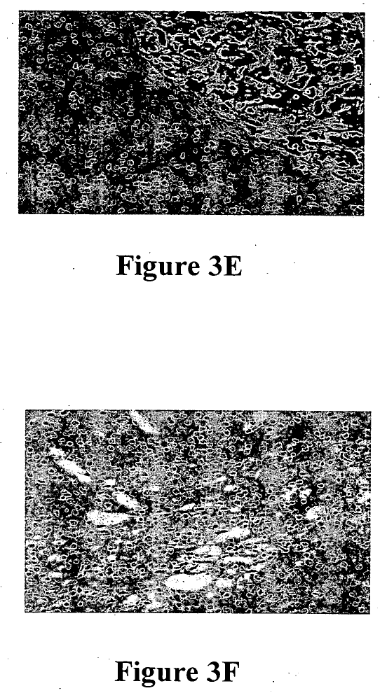

Paraffin-embedded tissue sections were stained with rat anti-LIMK1 monoclonal antibody. One the metastatic tumors but not normal tissues show high levels of LIMK1 protein seen as brown color (FIG. 3). In the figure, (A) is normal breast tissue; (B) is metastatic breast cancer, (C) is normal lung tissue, (D) is metastatic lung cancer; (E) is metastatic prostate cancer tissue (dark brown) and adjacent normal tissue; and (F) is isotype control of prostate tissue.

### EXAMPLE 11

### Search for LIMK1 Inhibitors

Inhibitors of LIMK1 activity, potentially useful in inhibiting or preventing in vivo invasiveness of inter alia metastatic breast and prostate cell lines are sought.

(a) Generation of Polyclonal Antibodies

Polyclonal antibodies that recognize phosphorylated LIMK1 are generated. These antibodies are used, in a high-throughput primary screen, to search for compounds that inhibit phosphorylation of LIM kinase. Cells expressing GFP-LIMK1 are plated on microtiter plates in the present or absence of inhibitors for 24 hours after which they are lysed. Flourescine-labeled anti-phospho LIMK1 antibodies are added and the change in flourescence (FRET) is measured. No changes in FRET will be observed if LIMK phosphorylation is inhibited and the antibody cannot bind to GFP-LIMK1.

(b) Validation via Secondary Screen

Positive “hits” are assayed for their ability to inhibit the activity of LIMK1; i.e. their ability to inhibit the invasiveness of metastatic breast and prostate cell lines in vitro, using methods similar to those described in Examples 2, 3 and 6, above. Once active molecules are identified, they are tested in the in vivo model of metastatic disease described in Examples 4 and 7, above.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adams et al., Cancer Res. 53: 4026-4034, 1993.
Brinkley et al., U.S. Pat. No. 5,326,692.
Chothia et al., J. Mol. Biol. 196: 901, 1987.
Chothia et al., J. Mol. Biol. 227: 799, 1992.
Chou et al. (U.S. Pat. No. 6,056,957).
Coligan et al., Current Protocols in Immunology, John Wiley & Sons, Inc., 1991′-1997.
Cumber et al., J. Immunol. 149: 120-126, 1992.
Davies & Riechmann, FEBS Lett. 339: 285-290, 1994.
Dower et al., International Publication No. WO 93/06121.
Erlich, eds., PCR Technology, Stockton Press, NY, 1989.
European Patent Publication No. 0 239 400.
Fodor et al., Science 251: 767-777, 1991.
Gefter et al., Somatic Cell Genet. 3: 231-236, 1977.
Glockshuber et al., Biochem. 29: 1363-1367, 1990.
Goeddel et al., Nucleic Acids Res. 8: 4057, 1980.
Hamers-Casterman et at, Nature 363: 446-448, 1993.
Jones et al., Nature 321: 522-525, 1986.
Kabat et al., “Sequences of Proteins of Immunological Interest”, U.S. Department of Health and Human Services, 1983.
Kazal et al., Nature Medicine 2: 753-759, 1996.
Kennet et al (eds) Monoclonal Antibodies and Hybridomas: A New Dimension in Biological Analyses, pp. 376-384, Plenum Press, New York, 1980.
Kohler and Milstein, Eur. J. Immunol. 6(7): 511-519, 1976.
Kohler and Milstein, Nature 256: 495-499, 1975.
Kostelny et al., J. Immunol. 148: 1547-1553, 1992.
Kozbor et al., Methods in Enzymology 121: 140, 1986.
Krebber et al., J. Immunol. Methods 201(1): 35-55, 1997.
Ku & Schutz, Proc. Natl. Acad. Sci. USA 92: 6552-6556, 1995.
Lawn et al., Nucleic Acids Res. 2: 6103, 1981;
Lewis et al., J. Pathol. 195: 66-71, 2001.
Liu et al., Proc. Natl. Acad. Sci. USA 84: 3439-3443, 1987.
Mackay et al., Nucleic Acids Res. 30(16): 1292-1305, 2002.
Morgan et al. (U.S. Pat. No. 6,180,377).
Mullis et al., Cold Spring Harbor Symp., Quant. Biol. 51: 263, 1987.
O'Reilly et al., Biotechniques 25: 824-830, 1998.
Plückthun et al., In Antibody engineering: A practical approach 203-252, 1996.
Plückthun, Biochem. 31: 1579-1584, 1992.
Queen et al. (U.S. Pat. No. 6,180,370).
Reiter et al., Biochem. 33: 5451-5459, 1994.
Reiter et al., Cancer Res. 54: 2714-2718, 1994.
Reiter et al., J. Biol. Chem. 269: 18327-18331, 1994.
Richmann et al., Nature 332: 323-327, 1988;
Shulman et al., Nature 276: 269-270, 1978;
Singer et al., U.S. Pat. No. 5,573,909.
Toyama et al., “Monoclonal Antibody, Experiment Manual”, published by Kodansha Scientific, 1987.
Trowbridge, J. Exp. Med. 148(1): 313-323, 1978.
Verhoeyen et al., Science 239: 1534-1536, 1988.
Volk et al., J. Virol. 42(1): 220-227, 1982.
Walker et al., J. Biochem. Mol. Toxicology 15(3): 121-127, 2001.
Ward et al., Nature 341: 544-546, 1989.
Webber et al., Mol. Immunol. 32: 249-258, 1995.
Winter and Milstein, Nature 349: 293, 1991,

1. A method for detecting an aberrant cell in a subject or in a biological sample from said subject, said method comprising contacting cells, cell extracts, serum or other sample from said subject or said biological sample with an immunointeractive molecule specific for a LIM kinase or antigenic portion thereof and screening for the level of immunointeractive molecule-LIM kinase complex formations wherein an elevated presence of said complex relative to a normal cell is indicative of an aberrant cell.

2. The method of claim 1 wherein the subject is a mammal.

3. The method of claim 2 wherein the subject is a human.

4. The method of any one of claims 1 to 3 wherein the immunointeractive molecule is an immunoglobulin.

5. The method of claim 4 wherein the immunoglobulin is a monoclonal antibody.

6. The method of claim 4 wherein the immunoglobulin is a polyclonal antibody.

7. The method of claim 1 wherein the LIM kinase is LIM kinase 1.

8. The method of claim 1 wherein the LIM kinase is LIM kinase 2.

9. A method for detecting an aberrant cell in a subject or in a biological sample from said subject, said method comprising screening the level of an expression product of a gene encoding a LIM kinase wherein an elevated level of said expression product compared to a normal cell is indicative of an aberrant cell.

10. The method of claim 9 wherein the subject is a mammal.

11. The method of claim 10 wherein the subject is a human.

12. The method of claim 9 wherein the LIM kinase is LIM kinase 1.

13. The method of claim 9 wherein the LIM kinase is LIM kinase 2.

14. The method of claim 9 wherein the expression product is mRNA or cDNA.

15. A method for diagnosing the presence of cancer or cancer-like growth in a subject, said method comprising contacting cells or cell extracts from said subject or a biological sample from said subject with a LIM kinase-binding effective amount of an antibody having specificity for said LIM kinase or an antigenic determinant or epitope thereon and then quantitatively or qualitatively determining the level of a LIM kinase-antibody complex wherein the presence of elevated levels of said complex compared to a normal cell is indicative of the presence of a cancer.

16. A method for diagnosing the presence of a cancer in a subject, said method comprising obtaining mRNA from cells of said subject or from a biological sample from said subject and optionally generating cDNA and contacting said mRNA or cDNA with a genetic probe capable of hybridizing to and/or amplifying all or part of a nucleotide sequence encoding LIM kinase or its complementary nucleotide sequence and then detecting the level of said mRNA or cDNA wherein the presence of elevated levels of said mRNA or cDNA compared to normal controls is indicative of the presence of cancer.

17. The method of claim 15 or 16 wherein the LIM kinase is LIM kinase 1.

18. The method of claim 15 or 16 wherein the LIM kinase is LIM kinase 2.

19. The method of claim 15 or 16 wherein the subject is a mammal.

20. The method of claim 19 wherein the mammal is a human.

21. The method of claim 16 wherein mRNA levels are determined by PCR.

22. The method of claim 21 wherein the PCR is real-time PCR.

23. A deimmunized antibody molecule having specificity for an epitope recognized by a monoclonal antibody to LIM kinase wherein at least one of the CDRs of the variable domain of said deimmunized antibody is derived from the said monoclonal antibody to LIM kinase and the remaining immunoglobulin-derived parts of the deimmunized antibody molecule are derived from an immunoglobulin or an analog thereof from the host for which the antibody is to be deimmunized.

24. A method for detecting cancer cells in a human patient, said method comprising introducing into said patient a deimmunized form of a non-human derived monoclonal antibody specific for human LIM kinase or an antigenic determinant thereon labeled with a reporter molecule, allowing dissemination of the labeled antibody throughout the circulatory system, or to selected parts of the circulatory system and then subjecting said patient to reporter molecule-detection means to identify the location of the antibody.

25. The method of claim 24 wherein the cancer comprises hyperplastic/neoplastic cells of hematopoietic origin.

26. The method of claim 25 wherein the cancer is selected from acute promyeloid leukemia, acute myelogenous leukemia, chronic myelogenous leukemia, acute lymphocytic leukemia, chronic lymphocytic leukemia, prolymphocytic leukemia, hairy cell leukemia or Wodenstrom's macroglobulinemia.

27. The method of claim 25 or 26 wherein the cancer is breast cancer.

28. The method of claim 25 or 26 wherein the cancer is in the lung, gastrointestinal trait or genito-urinary tract.

29. The method of claim 25 or 26 wherein the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcimona, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medullobalstoma, craniopharyngioma, ependymoma, pinealoma, hemangioblastoma, acoustic neuroma, oliogdendroglioma, meningioma, melanoma, neuroblastoma or retinoblastoma.

30. A method for the treatment of a patient having cancer, said method comprising administering to said human a cancer cell growth inhibiting-effective amount of an antibody having specificity for human LIM kinase and being substantially non-immunogenic and further comprising a cell growth inhibiting or cell killing agent fused, bound or otherwise associated thereto.

31. A method of treating a patient having cancer or a related condition, said method comprising the administration to said patient of a LIM kinase-inhibiting effective amount of an agent for a time and under conditions sufficient to inhibit the activity of LIM kinase or reduce levels of LIM kinase and to reduce cancer cell growth.

32. A composition comprising an agent capable of acting as a modulator of LIM kinase activity or gene expression and one or more pharmaceutically acceptable carriers and/or diluents.

33. A method for the treatment of a patient having cancer, said method comprising administering to said human, a genetic composition comprising a genetic construct which down-regulates expression of a gene encoding LIM kinase.

34. Use of a monoclonal antibody to LIM kinase in the manufacture of a quantitative or semi-quantitative diagnostic kit to determine relative levels of LIM kinase in suspected cancer cells from a patient.
